(19) United States
(12) Patent Application Publication
(10) Pub. No.: US 2021/0396767 A1
(43) Pub. Date: Dec. 23, 2021

(54) Method for Predicting Prognosis of Ischemic Disease

(71) Applicants: GACHON UNIVERSITY OF INDUSTRY-ACADEMIC COOPERATION FOUNDATION, Seongnam-si (KR); KOREA BASIC SCIENCE INSTITUTE, Yuseong-gu Daejeon (KR)

(72) Inventors: Bonghee LEE, Jeju-si (KR); Bayarsaikhan DELGER, Incheon (KR); Jaesuk LEE, Incheon (KR)

(73) Assignee: GACHON UNIVERSITY OF INDUSTRY-ACADEMIC COOPERATION FOUNDATION, Seongnam-si (KR)

(21) Appl. No.: 17/287,934

(22) PCT Filed: Oct. 25, 2019
(86) PCT No.: PCT/KR2019/014148

§ 371 Date: Apr. 22, 2021

(30) Foreign Application Priority Data
Oct. 25, 2018 (KR) 10-2018-0128270

(51) Int. Cl. G01N 33/68

(52) U.S. Cl. CPC G01N 33/6893; G01N 2333/765; G01N 2333/52; G01N 2800/52; G01N 2333/70503; G01N 2440/38

(57) ABSTRACT

The present invention relates to a method for predicting the prognosis of ischemic disease using an AGE-RAGE-based biomarker. The biomarker can be used as an indicator factor of the clinical severity of ischemic disease, can maximize the efficiency of stem-cell treatment by determination of the optimal timing of the stem-cell treatment on the basis of changes in the biomarker according to the severity of ischemic disease, and can also be used as a useful indicator factor for verifying efficacy after the stem-cell treatment.

### BACKGROUND OF THE INVENTION

### 1. Field of the Invention

The present invention relates to a method for predicting the prognosis of ischemic disease using an AGE(advanced glycation endproduct-protein)-RAGE(receptor-AGEs)-based biomarker.

The present invention also relates to a biomarker for verifying the optimal application time of a stem cell therapeutic agent for ischemic disease.

In addition, the present invention relates to a biomarker for analyzing the severity of ischemic disease.

### 2. Description of the Related Art

Ischemic disease can be induced by several causes. For example, ischemic symptoms may appear due to arteriosclerosis, hypotension, compression of blood vessels from outside the blood vessels (for example, compression of blood vessels by cancer outside the blood vessels), blood clots, embolism, accidental cerebrovascular damage, stroke, cerebral infarction, arteriovenous malformation, peripheral arterial occlusive disease, etc.

In the case of chronic ischemic heart disease, one of the representative ischemic diseases, symptoms such as chest pain, dyspnoea due to heart failure, weakness, and fainting due to an abnormality in the coronary circulatory system that supplies blood to the myocardium are accompanied, and the incidence rate is rapidly increasing in recent years. In addition, interruption of blood supply due to ischemia causes various ischemic diseases such as ischemic heart failure, ischemic enteritis, eye disease, and ischemic lower limb disease.

Treatment methods for these ischemic diseases include pharmacotherapy and coronary angioplasty that expands narrow blood vessels using stents, and sometimes arterial bypass is performed in the case of the heart. However, these treatment methods are not play a role as appropriate treatment methods if the blood vessels are too hard, all blood vessels that can be used for transplantation are used, or if there is continuous recurrence even after the repeated execution of arterioplasty due to restenosis. Therefore, a new method is required to overcome the limitations of such existing treatment methods, and gene therapy using angiogenic factors is attracting attention as a new treatment strategy. A less invasive method developed recently is therapeutic angiogenesis, which is a method by the introduction of a pro-angiogenic factor that relieves ischemia in order to improve the formation of new blood vessels in ischemic tissues. However, this method has not yet completely cured ischemic disease, and there is a problem with unexpected side effects.

On the other hand, in recent years, researches on cell therapy products for the treatment of ischemic diseases are increasing, and in particular, technologies using stem cells are being developed. Korean Patent Publication No. 2006-0091296 discloses a technology for treating ischemic diseases using embryonic stem cells.

Although various clinical applications using stem cell therapy have been attempted, development of a clinical biomarker for predicting the therapeutic efficiency of stem cell therapy in advance and verifying the therapeutic effect is insignificant. In the case of applying stem cell therapy to patients with severe lower limb ischemia and acute myocardial infarction as an ischemic disease, there is currently no biomarker to verify the effectiveness of treatment in patients under any conditions. In addition, since there are few objective disease-specific biomarkers that can verify the effectiveness after stem cell treatment, there is an urgent need for research on this.

AGE (advanced glycation end-product) is a complex that is produced constantly in the human body, and is mainly generated by the reaction of carbohydrates and free amino acids. It is known as a molecule that promotes the death of neurons because it is a chemically very unstable and highly reactive substance. In addition, AGE is reported to be increased in the brain of the elderly or aged animals, and it affects all cells and biomolecules, causing aging and aging-related chronic diseases. In other words, AGE is associated with adult diseases such as aging, Alzheimer's disease, kidney disease, diabetes, diabetic vascular complications, diabetic retinal abnormalities and diabetic neurological abnormalities by increasing vascular permeability, inhibiting vasodilation caused by obstruction of nitric oxide, oxidizing LDL, secreting various types of cytokines in macrophages or endothelial cells, and increasing oxidative stress.

In this study, the present inventors confirmed that activated macrophages migrated and secreted to the ischemic region, so that AGE was discharged and accumulated in the ischemic region and blood. The present inventors also confirmed that Receptor-AGEs (RAGE) were expressed in the membrane of myocardial/muscular cells and injected stem cells in the ischemic region, and the expressed cells were killed, and the increase of RAGE in the blood can be a prognostic indicator showing the state of apoptosis. The present inventors have confirmed that measurement of AGE-albumin-derived peptides and RAGE binding proteins such as S100-A8 and S100-A4 using mass spectrometry can be used for this purpose.

Accordingly, the present inventors tried to develop a biomarker capable of simultaneously predicting the efficacy of stem cells and the patient's prognosis by measuring the expression levels of AGE and RAGE in the blood after stem cell administration in patients with myocardial infarction and lower limb ischemia.

### SUMMARY OF THE INVENTION

It is an object of the present invention to provide a method for predicting the prognosis of ischemic disease using an AGE-RAGE-based biomarker.

To achieve the above object, the present invention provides a method of providing information for diagnosing the prognosis of ischemic disease comprising the following steps: 1) quantifying any one or more proteins selected from the group consisting of AGE-albumin, S100, HMGB1 and RAGE in a control sample isolated from a subject with a good prognosis and a sample isolated from a subject with suspected ischemic disease; 2) comparing the expression level of the protein of step 1); and 3) determining the ischemic disease patient with a bad prognosis if the protein expression level of the sample isolated from the subject with suspected ischemic disease was higher than that of the control sample in step 2).

### ADVANTAGEOUS EFFECT

The present invention relates to a method for predicting the prognosis of ischemic disease using an AGE-RAGE-based biomarker. The biomarker can be used as an indicator factor of the clinical severity of ischemic disease, can maximize the efficiency of stem-cell treatment by determination of the optimal timing of the stem-cell treatment on the basis of changes in the biomarker, and can also be used as a useful indicator factor for verifying the efficacy after the stem-cell treatment.

### BRIEF DESCRIPTION OF THE DRAWINGS

FIGS. 1a and 1b are diagrams expressing the AGE-RAGE expression pattern that it is possible to determine the treatment timing and efficacy of stem cells through the method of the present invention:

FIG. 1a: prediction of treatment timing;

FIG. 1b: prediction of treatment efficacy.

FIG. 2a is a diagram illustrating the change in appearance of the lower limb of a mouse model after the injection of human bone marrow-derived mesenchymal stem cells in a lower limb ischemia model.

FIG. 2b is a diagram illustrating the level of limb loss for 7 days after the injection of human bone marrow-derived mesenchymal stem cells in a lower limb ischemia model.

FIG. 2c is a diagram illustrating the ischemia score for 7 days after the injection of human bone marrow-derived mesenchymal stem cells in a lower limb ischemia model.

FIG. 2d is a diagram illustrating the modified ischemia score for 7 days after the injection of human bone marrow-derived mesenchymal stem cells in a lower limb ischemia model.

FIG. 3a is a diagram illustrating the change in appearance of the lower limb for 3 days in the control group and the stem cell injection experimental group after the injection of human adipose-derived mesenchymal stem cells in a lower limb ischemia model. (Tie: control, Tie+MSC: experimental group)

FIG. 3b is a diagram illustrating the state in which human adipose-derived mesenchymal stem cells were injected in a lower limb ischemia model.

FIG. 3c is a diagram illustrating the level of limb loss for 3 days after the injection of stem cells. (Control, Tie, Tie+MSC)

FIG. 3d is a diagram illustrating the ischemia score for 3 days after the injection of stem cells. (Control, Tie, Tie+MSC)

FIG. 3e is a diagram illustrating the modified ischemia score for 3 days after the injection of stem cells. (Control), Tie, Tie+MSC)

FIG. 4a is a diagram illustrating the number of apoptosis of cardiomyocytes according to time as determined by a microscope in a myocardial infarction model.

FIG. 4b is a diagram illustrating the percentage of TUNEL-positive cells over time in a myocardial infarction model.

FIG. 5 is a diagram illustrating the changes in RAGE expression in the heart tissue of a myocardial infarction model, confirmed by immunohistochemistry.

FIG. 6 is a diagram quantitatively confirming the increase of AGE-albumin in a myocardial infarction model.

FIG. 7 is a diagram quantitatively confirming the change in RAGE in a myocardial infarction model.

FIG. 8 is a diagram comparing the changes in AGE-albumin and RAGE in a myocardial infarction model.

FIG. 9a is a diagram illustrating the glycation (in vitro) due to an excessive amount of glucose for albumin protein in blood:

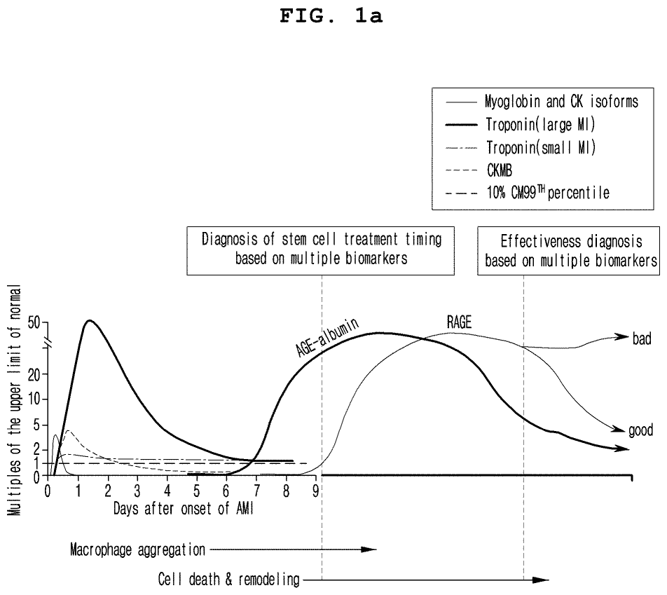

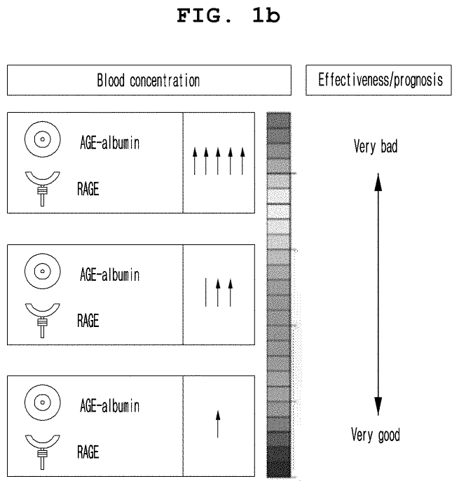

FL: fructosyl-lysine; a kind of primary glycation products of AGE-albumin;

CML+CMR: Carboxy Methyl Lysine+Carboxy Methyl Arginine; one of the secondary glycation forms of AGE-albumin, which is more toxic than the primary glycation and cannot be structurally reverted;

MG-H1: Methylglyoxal Hydro-limidazolone; one of the secondary glycation forms of AGE-albumin, which is more toxic than the primary glycation and cannot be structurally reverted;

CEL+CER: Carboxy Ethyl Lysine+Carboxy Ethyl Arginine; one of the secondary glycation forms of AGE-albumin, which is more toxic than the primary glycation and cannot be structurally reverted.

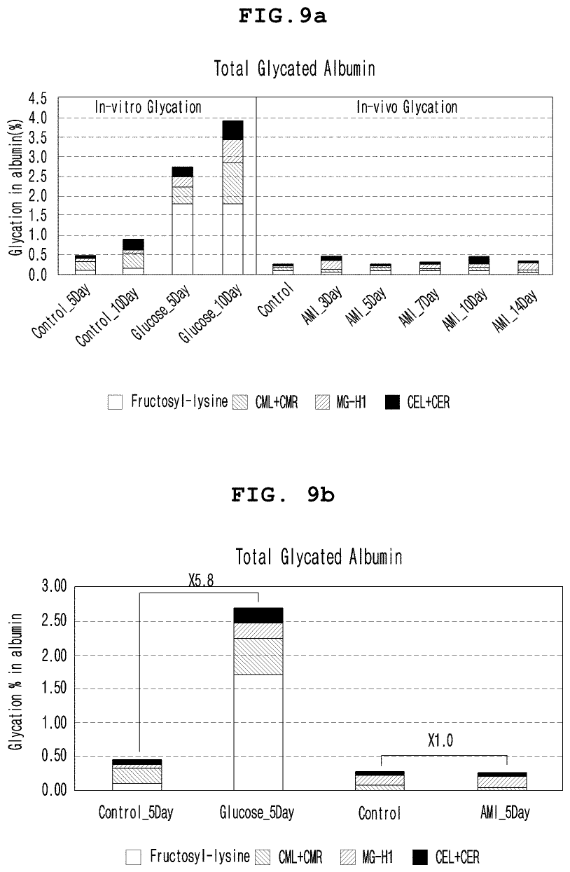

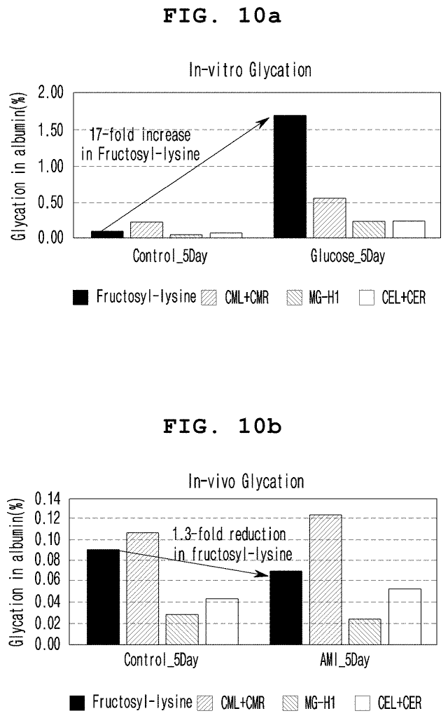

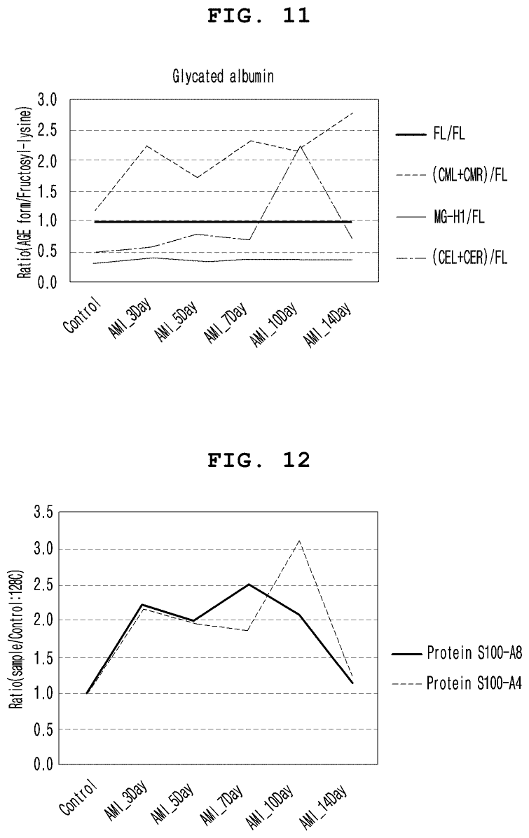

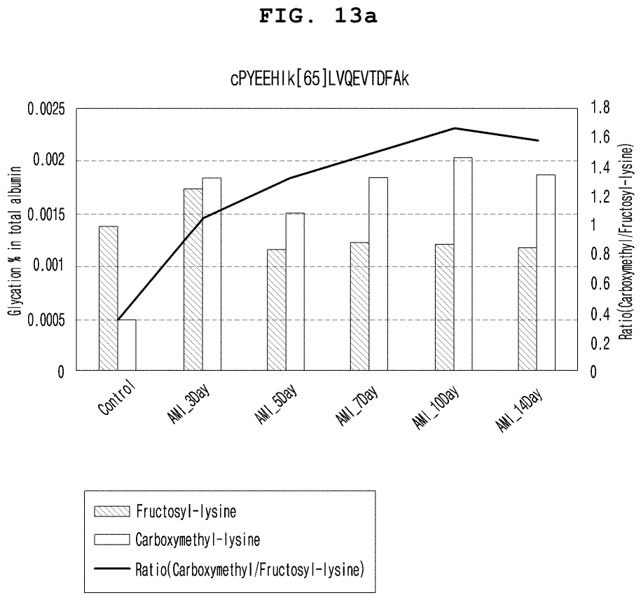

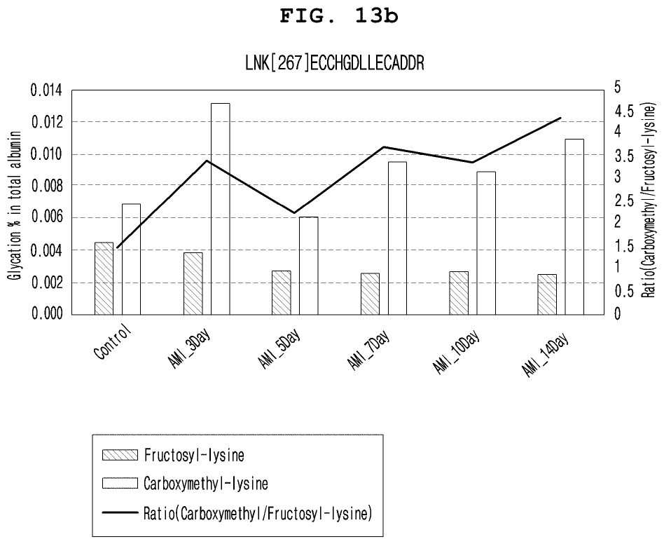

FIG. 9b is a diagram illustrating the glycation (in vivo) due to an excessive amount of glucose for albumin protein in blood:

FL: fructosyl-lysine; a kind of primary glycation products of AGE-albumin;

CML+CMR: Carboxy Methyl Lysine+Carboxy Methyl Arginine; one of the secondary glycation forms of AGE-albumin, which is more toxic than the primary glycation and cannot be structurally reverted;

MG-H1: Methylglyoxal Hydro-limidazolone; one of the secondary glycation forms of AGE-albumin, which is more toxic than the primary glycation and cannot be structurally reverted;

CEL+CER: Carboxy Ethyl Lysine+Carboxy Ethyl Arginine; one of the secondary glycation forms of AGE-albumin, which is more toxic than the primary glycation and cannot be structurally reverted.

FIG. 10a is a diagram illustrating the distribution of primary glycation products and advanced glycation end-products in vitro:

FL: fructosyl-lysine; a kind of primary glycation products of AGE-albumin;

CML+CMR: Carboxy Methyl Lysine+Carboxy Methyl Arginine; one of the secondary glycation forms of AGE-albumin, which is more toxic than the primary glycation and cannot be structurally reverted;

MG-H1: Methylglyoxal Hydro-limidazolone; one of the secondary glycation forms of AGE-albumin, which is more toxic than the primary glycation and cannot be structurally reverted;

CEL+CER: Carboxy Ethyl Lysine+Carboxy Ethyl Arginine; one of the secondary glycation forms of AGE-albumin, which is more toxic than the primary glycation and cannot be structurally reverted.

FIG. 10b is a diagram illustrating the distribution of primary glycation products and advanced glycation end-products in vivo:

FL: fructosyl-lysine; a kind of primary glycation products of AGE-albumin;

CML+CMR: Carboxy Methyl Lysine+Carboxy Methyl Arginine; one of the secondary glycation forms of AGE-albumin, which is more toxic than the primary glycation and cannot be structurally reverted;

MG-H1: Methylglyoxal Hydro-limidazolone; one of the secondary glycation forms of AGE-albumin, which is more toxic than the primary glycation and cannot be structurally reverted;

CEL+CER: Carboxy Ethyl Lysine+Carboxy Ethyl Arginine; one of the secondary glycation forms of AGE-albumin, which is more toxic than the primary glycation and cannot be structurally reverted.

FIG. 11 is a diagram illustrating the increase in the ratio of the advanced glycation end-product compared to the primary glycation product (FL) in the blood of a myocardial infarction model.

FIG. 12 is a diagram illustrating the changes of the expression levels of S100-A8 and S100-A4, the RAGE binding proteins, in the blood of a myocardial infarction model.

FIG. 13a is a diagram illustrating that the ratio of CML to FL was increased continuously from 3 to 14 days after the onset of myocardial infarction (AMI) caused by glycation that occurred at the 65^{th} lysine of the amino acid sequence of albumin.

FIG. 13b is a diagram illustrating that the ratio of CML to FL was increased continuously from 3 to 14 days after the onset of myocardial infarction (AMI) caused by glycation that occurred at the 267^{th} lysine of the amino acid sequence of albumin.

### DESCRIPTION OF THE PREFERRED EMBODIMENTS

In the present invention, the term “marker” refers to a molecule that is quantitatively or qualitatively associated with the presence of a biological phenomenon. The marker of the present invention refers to a gene that is a criterion for predicting a patient with a good or bad prognosis after the onset of ischemic disease.

In the present invention, the term “prognosis” means a prediction for medical consequences (eg, long-term viability, disease-free survival rate, etc.). The prognosis includes a positive prognosis (good prognosis) or a negative prognosis (bad prognosis), and the negative prognosis includes disease progression such as relapse and drug resistance or mortality, and the positive prognosis includes amelioration or stabilization of disease such as disease-free condition and disease improvement.

In the present invention, the term “prediction” means preliminary guessing about medical consequences. The prediction means, for the purpose of the present invention, to predict in advance the course of the disease (disease progression, amelioration, recurrence, drug resistance) in a patient diagnosed with ischemic disease.

Hereinafter, the present invention is described in detail.

The present invention provides a method of providing information for diagnosing the prognosis of ischemic disease comprising the following steps: 1) quantifying any one or more proteins selected from the group consisting of AGE-albumin, S100, HMGB1 and RAGE in a control sample isolated from a subject with a good prognosis and a sample isolated from a subject with suspected ischemic disease; 2) comparing the expression level of the protein of step 1); and 3) determining the ischemic disease patient with a bad prognosis if the protein expression level of the sample isolated from the subject with suspected ischemic disease was higher than that of the control sample in step 2).

The ischemic disease can be myocardial infarction or lower limb ischemia, and particularly, the myocardial infarction or lower limb ischemia can be acute myocardial infarction or severe lower limb ischemia, but not always limited thereto.

The sample of step 1) can be any one or more samples selected from the group consisting of whole blood, serum, plasma, saliva, urine, sputum, lymph fluid, cells, and tissues, but not always limited thereto.

The expression level of the protein can be confirmed by one or more methods selected from the group consisting of western blotting, enzyme-linked immunosorbent assay (ELISA), radioimmunoassay, radioimmunodiffusion, ouchterlony, rocket immunoelectrophoresis, immunohisto staining, immunoprecipitation assay, complete fixation assay, FACS (flow cytometry), protein chip assay and mass spectrometry, but not always limited thereto.

The RAGE can be specifically soluble RAGE, and the S100 can be specifically S100-A8 or S100-A4.

In addition, the present invention provides a kit for diagnosing or predicting the prognosis of ischemic disease comprising a quantification device for any one or more proteins selected from the group consisting of AGE-albumin, S100, HMGB1 and RAGE.

The ischemic disease can be myocardial infarction or lower limb ischemia, and particularly, the myocardial infarction or lower limb ischemia can be acute myocardial infarction or severe lower limb ischemia, but not always limited thereto.

The RAGE can be specifically soluble RAGE, and the S100 can be specifically S100-A8 or S100-A4.

The kit for predicting the prognosis of the present invention is composed of one or more other component compositions, solutions, or devices suitable for the analysis method.

For example, the kit of the present invention can be a kit comprising genomic DNA derived from a sample to be analyzed, a primer set specific for the marker gene of the present invention, an appropriate amount of DNA polymerase, dNTP mixture, PCR buffer, and water to perform PCR. The PCR buffer can contain KCl, Tris-HCl and MgCl₂. In addition, the constituents necessary for electrophoresis that can confirm whether the PCR product is amplified can be additionally included in the kit of the present invention.

In addition, the kit of the present invention can contain essential elements necessary to perform RT-PCR. The RT-PCR kit can include test tubes or other suitable containers, reaction buffers, deoxynucleotides (dNTPs), enzymes such as Taq-polymerase and reverse transcriptase, DNase, RNase inhibitors, DEPC-water and sterilized water, in addition to each primer set specific to a marker gene. In addition, the kit can include a primer set specific to a gene used as a quantitative control.

The kit of the present invention can be a kit containing essential elements necessary to perform DNA chip assay. The DNA chip kit can include a substrate to which cDNA corresponding to a gene or a fragment thereof is attached as a probe, and the substrate can include cDNA corresponding to a quantitative structural gene or a fragment thereof. In addition, the kit of the present invention can be in the form of a microarray having a substrate on which the marker gene of the present invention is immobilized.

The kit of the present invention can be a kit characterized by containing essential elements necessary to perform ELISA. The ELISA kit includes an antibody specific to a marker protein and an agent for measuring the protein level. The ELISA kit can include a reagent capable of detecting the antibody forming an “antigen-antibody complex”, for example, a labeled secondary antibody, chromopores, enzymes, and a substrate thereof. In addition, the kit can include an antibody specific to a quantitative control protein.

The term “antigen-antibody complex” used herein refers to a combination of a protein encoded by a gene and an antibody specific thereto. The amount of the antigen-antibody complex formation can be quantitatively measured through the signal level of a detection label. The detection label can be selected from the group consisting of enzymes, fluorescent substances, ligands, luminescent substances, microparticles, redox molecules, and radioisotopes, but not always limited thereto.

Hereinafter, the present invention will be described in detail by the following examples.

However, the following examples are only for illustrating the present invention, and the contents of the present invention are not limited thereto.

### EXAMPLE 1

### Construction of Myocardial Infarction Model

Acute myocardial infarction (AMI) model was constructed using Sprague-Dawley rats.

Particularly, Sprague-Dawley rats weighing 250 to 300 g were prepared and anesthetized with a mixture of Ketamine (50 mg/kg) and xylazine (4 mg/kg). A 16 gauge catheter was inserted into the trachea of the experimental animal and connected to an artificial respirator. The animal was laid on a flat plate and the limbs and tail were fixed with tape. The skin was incised vertically about 1 to 1.5 cm from the left side of the sternum. The gap between the pectoralis major muscle and the pectoralis minor muscle was separated and the 5^{th} intercostal space was confirmed. Then, the intercostal muscle was carefully incised about 1 cm horizontally. A retractor was placed between the fifth and sixth ribs and spread up and down. In normal rats, the thymus covers the upper part of the heart and blocks view, so the thymus was pulled toward the head using an angle hook. The shape of the left coronary artery was observed to determine which vascular branches to bind. Then, LAD (Left Anterior Descending artery) located 2-3 mm below the line where the pulmonary conus and the pointed part of the left atrial appendage intersect was tied with 6-0 silk. The opened fifth and sixth ribs were tightened again, and the incised intercostal muscle was tied with MAXON 4-0 filament. Then, the remaining air in the chest cavity was drained with a 23 gauge needle syringe so that the lungs could be fully extended. The skin was sutured using MAXON 4-0 filament, the tube that had been intubated into the trachea was removed, and the mucus in the pharynx was removed. After surgery, an analgesic (Buprenorphine 0.025 mg/kg) was injected subcutaneously into the experimental animal every 12 hours.

### EXAMPLE 2

### Construction of Lower Limb Ischemia Model

Modeling of a critical limb ischemia (CLI) model was performed by a method of blocking and releasing the blood flow in the femoral artery using mice. The prepared mice were anesthetized by intraperitoneal injection of 2% xylazine hydrochloride and tiletamin/zolazepam, and the left thigh was opened and the femoral artery was tied with 6.0 silk. Modeling of lower limb ischemia animals was performed by blocking the blood flow in the femoral artery for 1 hour and then allowing it to flow again.

### EXPERIMENTAL EXAMPLE 1

### Confirmation of Stem Cell Transplantation Effect in Lower Limb Ischemia Model

In the lower limb ischemia model, the effect of stem cell transplantation was confirmed.

Particularly, the muscle of the lower limb ischemia model constructed in Example 2 was treated with saline, 1×10⁶ bone marrow-derived mesenchymal stem cells or 1×10⁶ bone marrow-derived mesenchymal stem cells and 6.4 ug of sRAGE protein. The cells used above were grown in Dulbecco's modified Eagle's medium (DMEM, Gibco) supplemented with low glucose, 10% fetal bovine serum (FBS), and 1% gentamycin. After the treatment, the skin of the model was sutured using MAXON 4-0 filament, and an analgesic (Buprenorphine, 0.025 mg/kg) was injected subcutaneously every 12 hours. The limb loss, ischemia score, and modified ischemia score were checked for 7 or 3 days. The modified ischemia score was expressed by measuring the size of the fibrous tissue stained in blue by performing Masson's trichrome staining using Image J program. At this time, the stained sizes in each group were compared, and the rate of increase compared to the normal was expressed as a score.

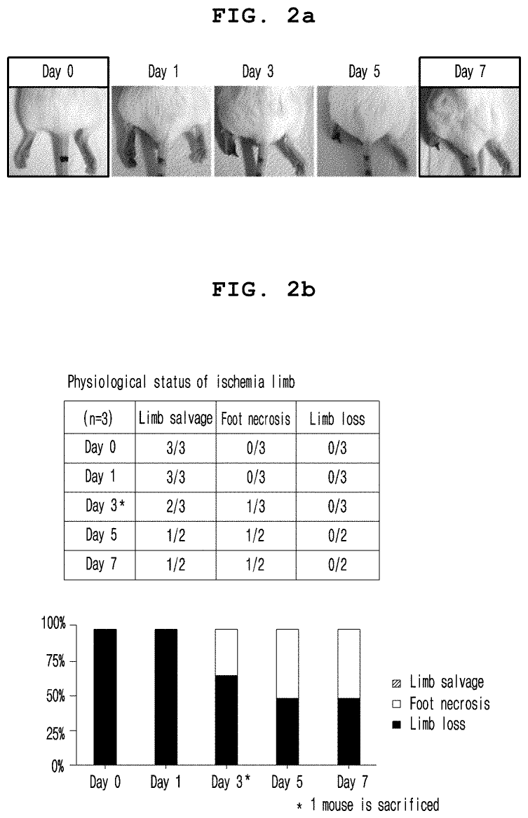

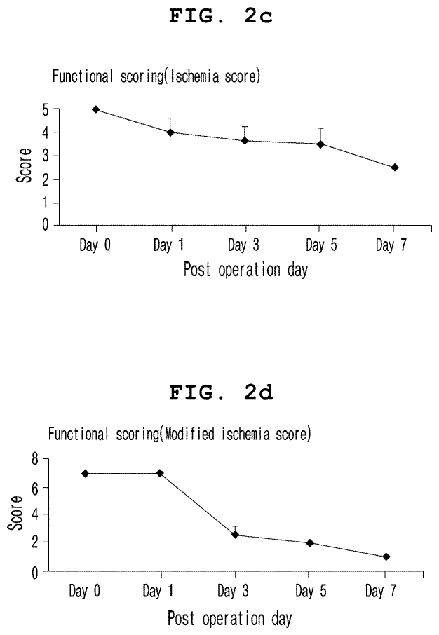

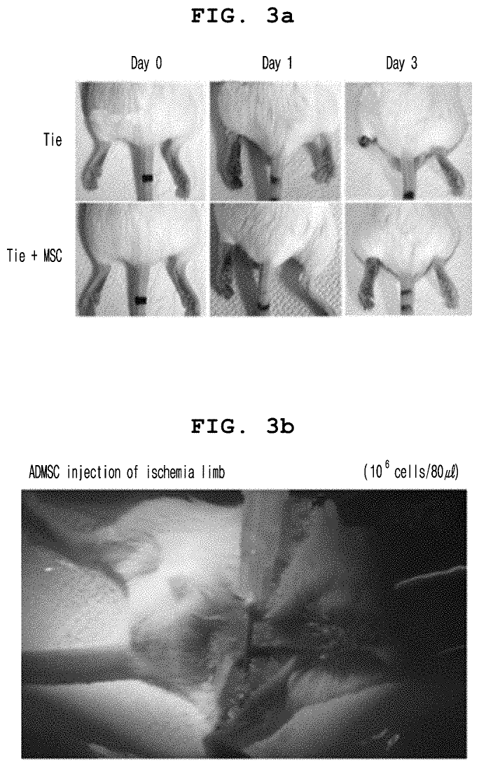

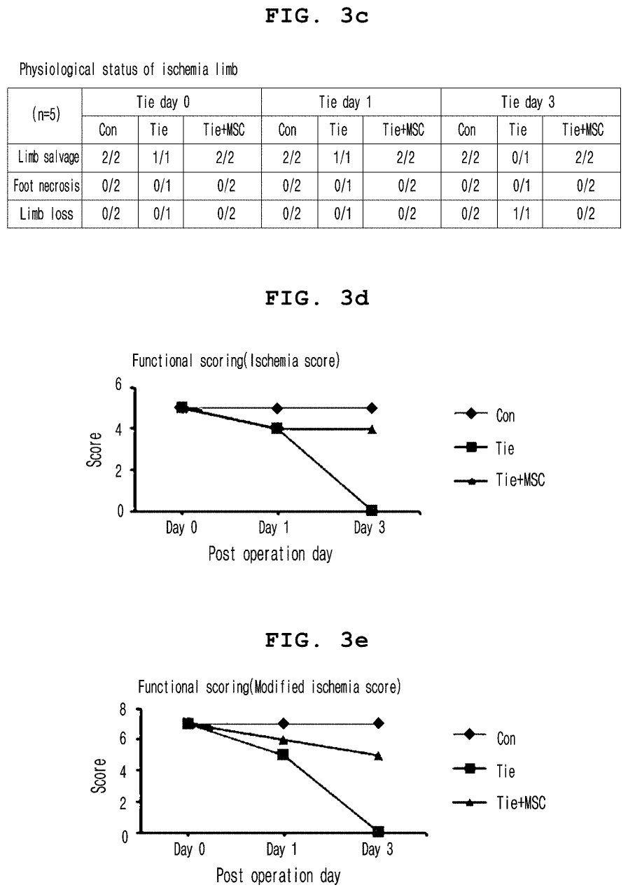

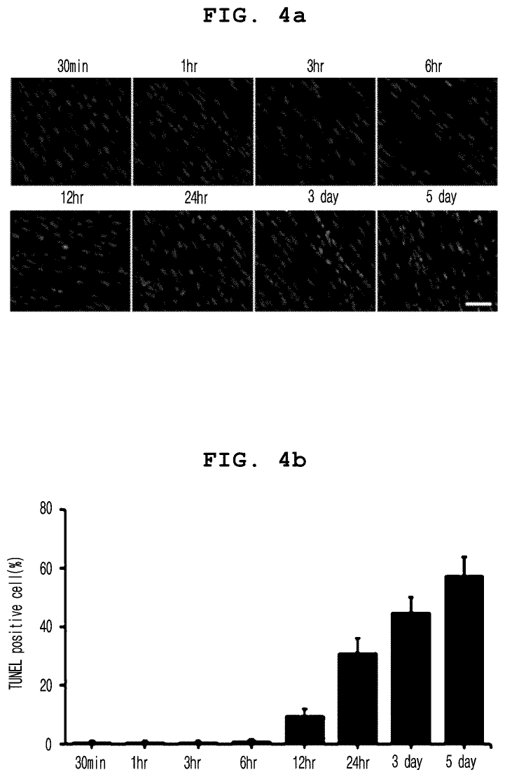

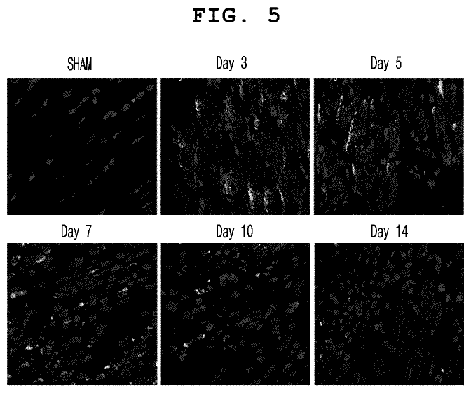

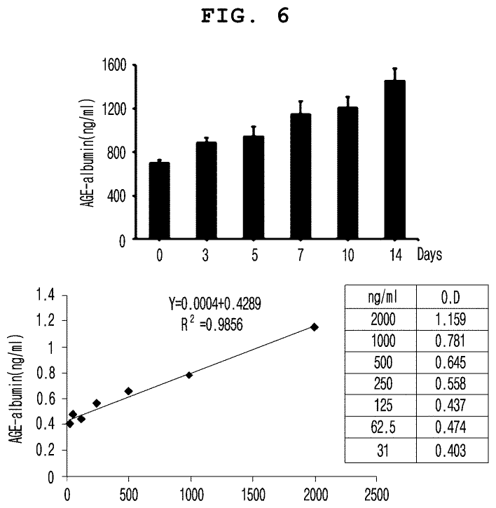

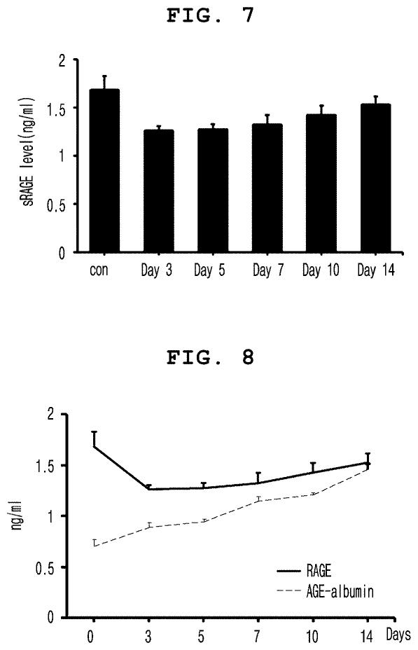

As a result, as shown in FIGS. 2a to 2d and 3a to 3e, the transplantation effect of the two stem cell lines was confirmed, and it was confirmed that the bone marrow-derived stem cells were excellent in relieving symptoms (FIGS. 2a to 2d and 3a to 3e).

### EXPERIMENTAL EXAMPLE 2

### Confirmation of Apoptosis, RAGE and AGE-Albumin Expression in Myocardial Infarction Model

### <2-1> Confirmation of Apoptosis

The myocardial tissue of the acute myocardial infarction model was made into paraffin blocks and TUNEL assay was performed. The deparaffinized tissues were treated with TUNEL reaction mixture (Roche, 12156792910) at 37° C. for 1 hour in a place with sufficient moisture and no direct sunlight. The tissues were washed 3 times with PBS and then observed under a microscope.

As a result, as shown in FIGS. 4a and 4b, it was confirmed that the number of apoptotic cardiomyocytes was increased over time (FIGS. 4a and 4b). In other words, it was confirmed that even when the blood supply was resumed, cardiomyocytes continued to die.

### <2-2> Confirmation of RAGE Expression

The expression of RAGE in the heart tissue of the acute myocardial infarction model was measured by immunohistochemistry. The deparaffinized myocardial tissues were washed with PBS and treated with a primary antibody at 4° C. overnight. The next day, the remaining primary antibody was washed off with PBS, and the tissues were treated with a secondary fluorescent antibody at room temperature for 1 hour. Finally, DAPI (4′6-diamino-2-phenylindole; 1 μg/ml, Invitrogen, D1306) was treated for 20 seconds to stain the nuclei and confirmed with a microscope.

As a result, as shown in FIG. 5, the expression level of RAGE was significantly increased until 3 to 7 days and then decreased again (FIG. 5).

The amount of soluble RAGE (sRAGE) in the acute myocardial infarction experimental group was measured using an ELISA kit (DuoSet ELISA, DY1616). A capture antibody was coated on a 96 well plate (100 ul/well) at 4° C. overnight. The next day, the remaining primary antibody was washed with PBS and the plate was treated with the serum of each group at 37° C. for 90 minutes. Then, the plate was washed again with PBS, and reacted with a detection antibody at room temperature for 2 hours. The plate was reacted with a horseradish peroxidase-conjugated secondary antibody at room temperature for 20 minutes. After reacting with 100 ul of a substrate solution, the optical density was measured at 450 nm to obtain the concentration of each sample.

As a result, as shown in FIG. 7, the amount of sRAGE measured on days 0-14 was decreased rapidly on the 3^{rd} day, but increased significantly until the 14^{th} day (FIG. 7).

### <2-3> Confirmation of AGE-Albumin

The amount of AGE-albumin was quantified by ELISA in the acute myocardial infarction experimental group. A 96 well plate was coated with 1 ug/ml of albumin antibody at 4° C. overnight. The next day, the remaining primary antibody was washed with PBS and the plate was treated with the serum of each group at 37° C. for 90 minutes. Then, the plate was washed again with PBS, and reacted with an AGE antibody at room temperature for 2 hours. The plate was reacted with a horseradish peroxidase-conjugated secondary antibody. The optical density of AGE-albumin was measured at 450 nm using TMB solution to obtain the concentration of each sample.

As a result, as shown in FIG. 6, the amount of AGE-albumin measured on days 0-14 was significantly increased (FIG. 6).

### EXPERIMENTAL EXAMPLE 3

### Confirmation of Type and Location of Glycation of Albumin Protein in Blood using Mass Spectrometry

### <3-1> Comparison of Glycation of Albumin Protein In Vitro and In Vivo

The glycation of albumin protein in blood was confirmed by mass spectrometry. 1 to 2 uL of a sample was taken and subjected to protein denaturing, disulfide bond degradation, and cysteine methylation, and then hydrolyzed using trypsin for more than 16 hours. The resulting peptide mixture after the hydrolysis was analyzed using Nanoflow LC-ESI/MS-MS. The results of comparing the glycation (in vitro) due to excessive glucose and the glycation (in vivo) due to myocardial infarction of albumin protein in blood are shown.

As shown in FIGS. 9a and 9b, In the case of the in-vitro experiment of glycation due to glucose, albumin total glycation was significantly increased to 4 to 5 times that of the control group, but in the case of the acute myocardial infarction model, it was hardly changed until 14 days as well as in the control group (FIGS. 9a and 9b).

### <3-2> Confirmation of Glycation Form

Glycated albumin was classified and compared by the form of glycation. As a result, it was confirmed that in vitro glycation and in vivo glycation were very different. In particular, fructosyl-lysine (FL), the form of primary (initial) glycation in vitro, was increased 17-fold over 5 days, but decreased by 1.3-fold in vivo (FIGS. 10a and 10b).

In vivo AGE glycation was compared based on FL. As a result, among the AGE glycation, the ratio of Carboxy Methyl Lysine (or arginine) (CML®) was high, which was continuously increased in the acute myocardial infarction model (FIG. 11). This result was consistent with the result of statistically significant increase in the amount of AGE-albumin measured using ELISA 0-14 days after the construction of ischemia model.

### <3-3> Confirmation of S100-A8 and S100-A4 Protein Expression

After constructing an acute myocardial infarction model, the RAGE binding protein S100 was detected in the blood of the experimental group. As a result, S100-A8 and S100-A4 proteins were observed to increase until 3 to 10 days (FIG. 12).

In tissues, the RAGE expression was shown to increase until day 7. This result showed the same tendency as the result that the RAGE expression level was significantly increased until 3 to 7 days and then decreased again as a result of measuring the RAGE expression in the heart tissue of the acute myocardial infarction model by immunohistochemistry (FIG. 5).

When observed by microscopic staining in tissues, reporters were increased by the AGE-albumin secreted from macrophages in cardiomyocytes, which represented an indicator of death of cardiomyocytes. This increase indicates that the index of cardiomyocyte death increases until the 7^{th} day.

### <3-4> Confirmation of Form and Location of Albumin Protein Glycation

It was observed that after the onset of myocardial infarction (AMI), the ratio of CML to FL in glycation of lysine of the amino acid sequence of albumin was increased continuously from 3 to 14 days (FIGS. 13a and 13b). Therefore, during the glycation of the peptide produced after hydrolysis of the glycated albumin, the increase of CML form indicates the increase of AGE-albumin, which is related to apoptosis, and this can be expressed as the ratio between FL form and CML form of the peptide glycated.

Therefore, the ratio of AGE-albumin glycation in the form of FL and CML, and S100 A8 and A4 proteins can be configured as a marker panel and used as a marker for diagnosing the prognosis of myocardial infarction or lower limb ischemia.

1.-10. (canceled)

11. A method of providing information for the prognosis of ischemic disease comprising the following steps:
1) quantifying any one or more proteins selected from the group consisting of AGE-albumin, S100, HMGB1 (high-mobility group box-1) and RAGE in a control sample isolated from a subject with a good prognosis and a sample isolated from a subject with suspected ischemic disease;
2) comparing an expression level of the protein of step 1); and
3) determining the ischemic disease patient with a bad prognosis if the protein expression level of the sample isolated from the subject with suspected ischemic disease was higher than that of the control sample in step 2).

12. The method of providing information for the prognosis of ischemic disease according to claim 11, wherein the ischemic disease is myocardial infarction or lower limb ischemia.

13. The method of providing information for the prognosis of ischemic disease according to claim 12, wherein the myocardial infarction or lower limb ischemia is acute myocardial infarction or severe lower limb ischemia.

14. The method of providing information for the prognosis of ischemic disease according to claim 11, wherein the RAGE is soluble RAGE.

15. The method of providing information for the prognosis of ischemic disease according to claim 11, wherein the S100 is S100-A8 or S100-A4.

16. The method of providing information for the prognosis of ischemic disease according to claim 11, wherein the samples are any one or more samples selected from the group consisting of whole blood, serum, plasma, saliva, urine, sputum, lymph fluid, cells and tissues.

17. The method of providing information for the prognosis of ischemic disease according to claim 11, wherein the expression level of the protein is confirmed by one or more methods selected from the group consisting of western blotting, enzyme-linked immunosorbent assay (ELISA), radioimmunoassay, radioimmunodiffusion, ouchterlony, rocket immunoelectrophoresis, immunohisto staining, immunoprecipitation assay, complete fixation assay, FACS (flow cytometry), protein chip assay and mass spectrometry.

18. The method of providing information for the prognosis of ischemic disease according to claim 11, further comprising a step of measuring a ratio of carboxymethyl-lysine (CML) form to fructosyl-lysine (FL) form in glycation of the peptides produced by hydrolysis of AGE-albumin.

19. A kit for diagnosing or predicting the prognosis of ischemic disease comprising a quantification device for any one or more proteins selected from the group consisting of AGE-albumin, S100, HMGB1 and RAGE.

20. The kit for diagnosing or predicting the prognosis of ischemic disease according to claim 19, wherein the S100 is S100-A8 or S100-A4.
